(19) United States
(12) Patent Application Publication
(10) Pub. No.: US 2025/0078964 A1
(43) Pub. Date: Mar. 6, 2025

(54) SYSTEMS AND METHODS FOR IDENTIFYING CANDIDATES FOR A TRIAL IN REAL TIME

(71) Applicant: LABORATORY CORPORATION OF AMERICA HOLDINGS, Burlington, NC (US)

(72) Inventors: Jennifer ENNIS, Chicago, IL (US); Chris STANLEY, Raleigh, NC (US); Enno DE VRIES, Burlington, NC (US); Greg GARRAWAY, Mebane, NC (US); Adam PLOTTS, Holly Springs, NC (US); Kristen ANDREWS, Genoa, IL (US); Gaurav SHARMA, High Point, NC (US); Aymeric GRASSART, Chicago, IL (US)

(73) Assignee: Laboratory Corporation of America Holdings, Burlington, NC (US)

(21) Appl. No.: 18/797,004

(22) Filed: Aug. 7, 2024

Related U.S. Application Data
(60) Provisional application No. 63/579,470, filed on Aug. 29, 2023.

(51) Int. Cl. G16H 10/20; G16H 10/60; G16H 15/00

(52) U.S. Cl. CPC G16H 10/20; G16H 10/60; G16H 15/00

(57) ABSTRACT

Systems and methods can evaluate candidates for eligibility to take part in trials. For example, a system may receive a request related to a candidate and access a database that includes a collection of data related to the candidate. In addition, the system may receive an update to the collection of data to generate an updated collection of data and automatically evaluate the updated collection of data against a plurality of trial criteria. In some examples, the system may determining at least one potential trial for the candidate based on the evaluation and convert the updated collection of data into one or more criteria sections and may subsequently automatically generate, substantially contemporaneous to determining the at least one potential trial, a graphical user interface (GUI) including the one or more criteria sections and automatically display, within the GUI, a message to one or more users related to eligibility of the candidate for the at least one potential trial.

### CROSS-REFERENCE TO RELATED APPLICATION

The present application claims the benefit of priority to U.S. Provisional Application No. 63/579,470, filed Aug. 29, 2023, the entire contents of which are incorporated by reference herein for all purposes.

### FIELD

The present disclosure relates to a method and system for identifying candidates for a trial, particularly, identifying eligible candidates for a trial in real-time.

### BACKGROUND

Currently there is a shift from analog records to digital records for ease of access and analysis. The transition of records and requirements for how electronic records can be stored varies by industry. For example, in the medical field, electronic health records (EHR) have been created to maintain candidate records. The EHRs are the collection of candidate information electronically stored in a digital format. However, individual hospitals and hospital partnerships each have their own EHR systems and collection of EHRs.

Frequently, candidates have information stored across a variety of EHRs as well as other data service providers. For example, whenever a hospital commissions a test to be performed for a candidate, such as a bloodwork panel, the test results are stored in the system utilized by the laboratory. This can include an onsite laboratory sharing usage of the EHR or the laboratory can be a third part laboratory having their own data storage system. With candidate data spread across multiple, often disparate systems, it can be difficult for a trial coordinator to analyze all of the data in convenient and meaningful manner. For example, the candidate data related to the trial coordinator's medical facility can be displayed in one format, test results from laboratories and third-party laboratories may not be compatible with the EHR such that they are provided in paper or other electronic format (e.g., printable document files). Additionally, the data from different sources, or even the same source, cannot be provided in a customizable manner that allows a user to view data in a specified manner or manipulate the data for their particular purposes.

### SUMMARY

Candidates can be evaluated in real-time for eligibility to take part in trials. For example, a method described herein can include receiving a request related to a candidate. Additionally, the method can include accessing a database that includes a collection of data related to the candidate. The method can further include receiving an update to the collection of data to generate an updated collection of data and evaluating the updated collection of data against a plurality of trial criteria. In some examples, the method may include automatically evaluating the updated collection of data against a plurality of trial criteria. The method can include determining at least one potential trial for the candidate based on the evaluation and converting the updated collection of data into one or more criteria sections. The method can also include automatically generating, substantially contemporaneously to determining the at least one potential trial, a graphical user interface including the one or more criteria sections. In some examples, the method may include automatically displaying a message to one or more users related to eligibility of the candidate for the at least one potential trial.

In another example, automatically generating the GUI further includes generating a trials dashboard tab within the GUI and populating, automatically and substantially contemporaneously to determining the at least one potential trial, the one or more criteria sections regarding the at least one potential trial on the trials dashboard tab.

In another example, evaluating the updated collection of data against the plurality of trial criteria may further include producing, for each of the at least one potential trial, a completion score for the candidate. The method may further include receiving, via the trials dashboard tab, additional information regarding qualifications of the candidate relevant to criteria for the at least one potential trial. In some examples, the method may further include updating the completion score associated with the at least one potential trial.

In another example, the method may further include receiving, for each of the at least one potential trial, a response regarding candidate interest in a potential trial.

In another example, for each response that indicates that the candidate is not interested in the potential trial, the method may further include removing the information regarding the potential trial associated with the response and may include updating the updated collection of data with the response regarding the candidate interest in the potential trial.

In another example, for each response that indicates that the candidate is interested in the potential trial, the method may further include exporting the information regarding the potential trial associated with the response into a trial candidate module.

In another example, the method may include importing, automatically, additional information regarding a potential trial into the trial candidate module such that the additional information related to an available candidate that has expressed interest in at least one other trial similar to the potential trial.

In another example, a system described herein can include one or more processors. The system can also include one or more memories. The one or more memories can include instructions executable by the one or more processors to perform operations. The operations can include receiving a request for data related to a candidate. Additionally, the operations can include accessing a database that includes a collection of data related to the candidate. The operations can further include evaluating the collection of data against a plurality of trial criteria. The operations can include determining at least one potential trial for the candidate. The operations can also include presenting, substantially contemporaneously to determining the at least one potential trial, information regarding the at least one potential trial.

In another example, a non-transitory computer-readable medium described herein can include instructions that are executable by one or more processing devices for causing the one or more processing devices to perform operations. The operations can include receiving a request for data related to a candidate. Additionally, the operations can include accessing a database that includes a collection of data related to the candidate. The operations can further include evaluating the collection of data against a plurality of trial criteria. The operations can include determining at least one potential trial for the candidate. The operations can also include presenting, substantially contemporaneously to determining the at least one potential trial, information regarding the at least one potential trial.

### BRIEF DESCRIPTION OF THE DRAWINGS

FIG. 1A depicts an illustrative system for implementing steps in accordance with the aspects of the present disclosure.

FIG. 1B is a block diagram illustrating some functional components for providing trial eligibility processing.

FIG. 2A is an example of a first portion of a trials tab of a graphical user interface (GUI) for assisting a physician in identifying possible trial opportunities for a candidate during a candidate encounter in accordance with various embodiments.

FIG. 2B is an example of a second portion of a trials tab of a GUI for assisting a physician in identifying possible trial opportunities for a candidate during a candidate encounter in accordance with various embodiments.

FIG. 2C is an example of a third portion of a trials tab of a GUI for assisting a physician in identifying possible trial opportunities for a candidate during a candidate encounter in accordance with various embodiments.

FIG. 3A is an example of a login GUI for a trial candidate module to assist authorized users with identifying candidates in trials in accordance with various embodiments.

FIG. 3B is an example of a candidate search GUI in organization search mode for a trial candidate module to assist authorized users with identifying candidates in trials in accordance with various embodiments.

FIG. 3C is an example of a candidate search GUI in candidate name search mode for a trial candidate module to assist authorized users with identifying candidates in trials in accordance with various embodiments.

FIG. 3D is an example of a candidate list GUI for a trial candidate module to assist authorized users with identifying candidates in trials in accordance with various embodiments.

FIG. 3E illustrates examples of a filter feature and an action feature included in a candidate list GUI for a trial candidate module to assist authorized users with identifying candidates in trials in accordance with various embodiments.

FIG. 3F is an example of a candidate detail GUI for a trial candidate module to assist authorized users with identifying candidates in trials in accordance with various embodiments.

FIG. 3G is an additional example of a candidate detail GUI for a trial candidate module to assist authorized users with identifying candidates in trials in accordance with various embodiments.

FIG. 4 is a flow diagram for generating a trials tab for assisting a physician in identifying possible trial opportunities for a candidate during a candidate encounter in accordance with various embodiments.

FIG. 5 block diagram of a computing device for implementing steps in accordance with the aspects of the present disclosure.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs. Frequently, EHRs and other types of trial records have different phases to support trials. These different support phases can include finding a suitable protocol, finding a suitable collection of candidates, finding suitable investigators, and locating candidates willing to enroll in trials. Speed to market can be critical. A rapid identification of candidates to start protocol for the trial can be beneficial. For example, in clinical trial applications in a medical field, the rapid identification of candidates can improve chances of an early approval for pharmaceuticals associated with the clinical trial. Databases that include information relevant to potential candidates for the trial can become stale within 24-48 hours. In addition, identifying and presenting potential candidates provides a significant technical improvement compared to conventional applications since the potential candidate pool may be updated with new data in real time. For example, if a potential candidate shows interest in a particular trial, the application may update the database to ensure that the candidate is considered for the trial. In other examples, or in addition, if the potential candidate expresses a desire to not participate in a trial, the potential candidate may remove themselves from the candidate pool thus reducing the candidate pool size and making finding the right candidate easier for medical professionals.

The present disclosure relates to a method and system for evaluating candidates for eligibility to take part in one or more trials. The present disclosure is designed to collect and/or access data for a combination of local and disparate data sources, organize the data in a meaningful manner, and determine eligibility of a candidate for one or more trials during a candidate visit. In operation, a trial coordinator can meet with a candidate and may access candidate records during the visit. Accessing the candidate records can cause a background eligibility process to trigger to compare the candidate information to the criteria outlined in various trials. If a candidate is a match to a trial, the trial coordinator can be contacted to let the candidate know that they may be eligible for that trial. The match notification may also include providing additional information about the trial. The additional information can include details about the trial, candidate data related to criteria for the trial, and information that can be conveyed to the candidate. The candidate data related to criteria can include a summary of the criteria that the candidate meets, criteria that the candidate does not meet, and unknown criteria, meaning that the candidate may or may not meet the unknown criteria depending on supplemental information that is currently not in the database.

The eligibility process can include identifying partial or potential matches and sending requests to the trial coordinator to obtain additional data. For example, the eligibility process can include a request to query the candidate for supplemental information to help determine if the candidate meets unknown criteria. The eligibility process can query the trial coordinator for additional information from the candidate prior to making an eligibility determination. The eligibility process can use any combination of local system data and local or remote database data when making determinations. In some embodiments, the eligibility process may not include geographic limitations. A candidate living in New York state may be considered for a trial that includes a principal investigator based in California. The additional data obtained from the trial coordinator can include a referral of the candidate to the trial by the trial coordinator. In a non-limiting example, the trial coordinator can refer the candidate to the trial and maintain a position as a specialist, principal investigator, and/or a primary physician to the candidate. The candidate can participate in the trial without changing physicians or specialists and can maintain a connection with a current physician or specialist.

In conventional systems, there can be an abundance of information, however, there may not be a mechanism provided to convey the information to a user in a combined manner. Typically, information can be viewed independently from one another in its original format. For example, a diagnostic test result for a candidate can be viewed as an independent reporting but it is not typically viewable as it relates to previous test results of the same type; much less as it relates to previous test results of other but potentially related types of tests. Often, the medium for accessing the information is limited in its ability to convey the information in any format other than how it was originally received. For example, EMRs or EHRs for a record management system 104 may only be able to display information as it was received from internal or external laboratories provided reports. A system of the present disclosure provides an improved graphical user interface, system, and methodology for combining, organizing, and visualizing data associated with trial eligibility of a candidate to a user in a meaningful way. The system can continuously receive information and updates regarding trials. For example, the information can be received from medical practitioners associated with trials such as clinical research coordinators, investigators, principal investigators, clinical research associates, clinical researchers, etc. The information can involve a direct input into a user interface for the system, imported from various databases, converted into formatted data from natural language inputs submitted to the user interface by employing natural language processing (NLP), etc.

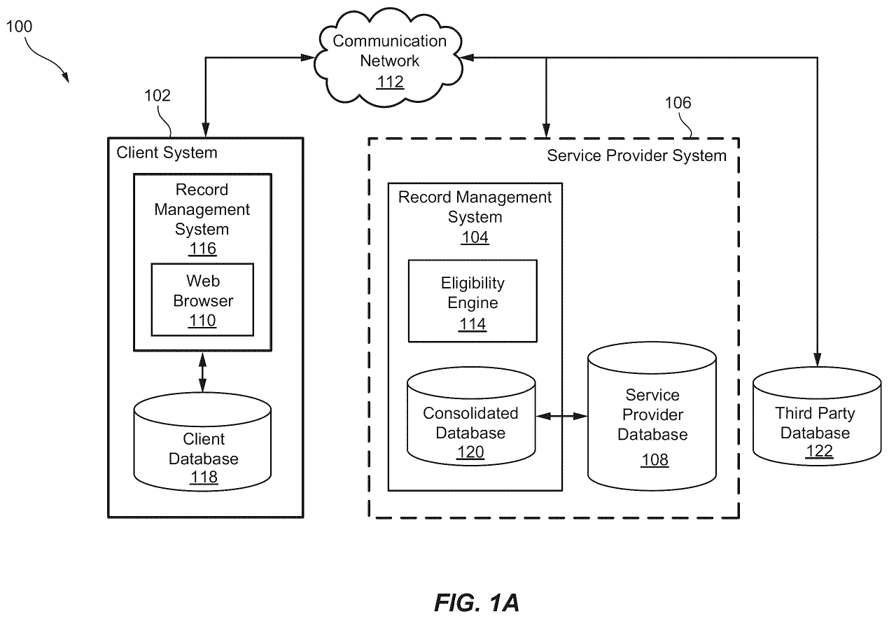

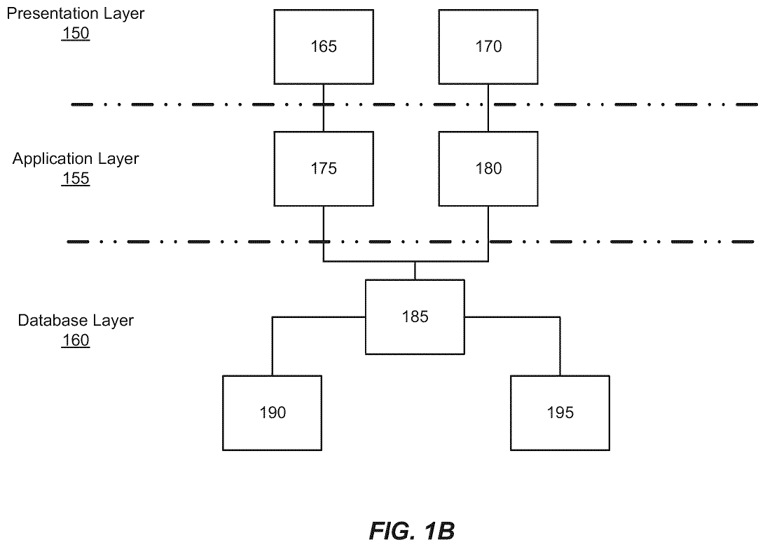

FIG. 1A depicts an example of a system 100 for implementing the steps in accordance with the aspects of the present disclosure. In particular, FIG. 1A depicts a system 100 including a client system 102, a record management system 104, and a service provider system 106. The record management system 104 can be part of the service provider system 106 or a separate system and/or service provider. Each of the client system 102, the record management system 104, and the service provider system 106 can include a combination of hardware and software configured to carry out aspects of the present disclosure. In particular, the client system 102, the record management system 104, and the service provider system 106 can include a computing system with specialized software and databases designed for providing a method for determining eligibility for a trial. As would be appreciated by one skilled in the art, each of the client system 102, the record management system 104, and the service provider system 106 can include a single computing device, a collection of computing devices in a network computing system, a cloud computing infrastructure, or a combination thereof. For example, the computing devices can include any combination of servers, personal computers, laptops, tablets, smartphones, etc. In some embodiments, different components or elements of the client system 102, the record management system 104, and the service provider system 106 can include their own computing devices.

In some embodiments, the client system 102 can include a computing device having software installed thereon for accessing data provided by the other components within the system 100. For example, the client system 102 can include a computing device having a web browser 110 configured to access web pages and downloading content on a network such as the communication network 112 (e.g., Internet). In some embodiments, the client system 102 can include or otherwise can be communicatively coupled to a record management system 116 and a client database 118. The record management system 116 and the client database 118 can be designed to manage and store records for access by the client system 102. The records can include any suitable combination of records, including, but not limited to, electronic medical records (EMR) and/or electronic health records (EHR) and software to manage and access the EMR/HER and/or records related to the candidate and relevant to the trial (e.g., nutrition records, hereditary records, etc.). In an example, the client system 102 can be a system operated by a medical service provider and the records stored within the client database 118 can be candidate records for that medical service provider. Similarly, the client system 102 can also be configured to be communicatively coupled to the record management system 104 and/or the service provider system 106 and can include software for accessing, reading, writing, etc. data on the record management system 104 and/or the service provider system 106. The client system 102 can either access the record management system 104 through the communication network 112, it can be directly coupled/networked to the record management system 104, service provider system 106, or both.

Similar to the client system 102, the service provider system 106 can include any combination of systems that host third party applications or services to be utilized by the client system 102, for example, as described in more detail in provisional patent application 63/387,859, which is incorporated by reference herein in its entirety. In some embodiments, the service provider system 106 can be a system of a clinical laboratory where tests are carried out on clinical specimens to obtain information about the health of a candidate to aid in diagnosis, treatment, and prevention of disease. For example, the tests can include medical tests to evaluate a person's physical or mental health such as tests on clinical specimens such as, but not limited to, blood or urine samples, tests on tissue specimens, tests on other biological materials, psychological or physiological evaluation, physical fitness, etc. The tests can be conducted using any combination of systems or methods. For example, the tests can include physical measurement, electromechanical analysis, analytical chemistry, molecular diagnostics, user evaluation, microbiology, serology or immunology, medical imaging, and the like. For purposes of this disclosure, the term tests will be used synonymously with any type of clinical laboratory test.

In some embodiments, the service provider system 106 can be a laboratory that includes an accessioning and processing system for receiving, sorting, and processing the one or more specimen/samples for testing purposes. All of the received, stored, and derived information related to accession data, candidate data, provider data, etc. can be stored in the service provider database 108. In some embodiments, each diagnostic testing order, including one or more tests on a specimen/sample, is linked to an accession identifier. Similar to the client database 118, the service provider database 108 can include any combination of computing devices configured to store and organize a collection of data. In some embodiments, the service provider system 106 can aggregate and store test data from a plurality of disparate data sources to be conveyed to a client system 102.

In some embodiments, and once all or a portion of the testing associated with an accession identifier is complete, the service provider system 106 can provide the results to the requesting entity. For example, if a user on the client system 102 submitted the diagnostic testing order to the laboratory, such as via service provider system 106, then the results can be provided to the user. The results can be provided to the requesting user using any combination of media. For example, the results can be provided via a web application interface, email, through the web browser 110, web application, a short message service (SMS), an app push notification, traditional mail, facsimile, etc. The results include, but are not limited to, any suitable combination of test results, test panel results, test chemistry results, analytic results, diagnostic procedures results, etc. Additionally, the results can include various levels of details depending on a type of tests being performed and information being conveyed. For example, results can include laboratory results, physiological results, mental results, physical results, etc. Additionally, the results can be conveyed using any suitable combination of informational presentations. For example, the results can be any suitable combination of measured values, expected values, graphs, color coding, detailed descriptions, etc. For purposes of this disclosure, the results will be used synonymously with any of these types of results.

The service provider system 106 can include or otherwise be communicatively coupled to the record management system 104. If separate from the service provider system 106, such as when the record management system 104 or a portion thereof is maintained by an entity (e.g., a medical care facility) separate from the service provider, the record management system 104 and the consolidated database 120 can share and/or receive some data with third parties, such as the service provider system 106. In some embodiments, the record management system 104 and the consolidated database 120 can aggregate, update, and deduplicate data from a collection of databases, for example, any combination of the client database 118, the service provider database 108, and any third-party sources such as third-party database 122. The data aggregation and deduplication can be performed using any combination of techniques, for example, as described in more detail in provisional patent application 63/387,859, which is incorporated by reference herein in its entirety. The record management system 104 can interact with the consolidated database 120 utilizing a given database model configured to interact with a user for analyzing the stored and consolidated data. The consolidated database 120 can include any combination of computing devices configured to store, update, and organize a collection of data. For example, consolidated database 120 can be a local storage device on the service provider system 106, a remote database facility, or a cloud computing storage environment.

In some embodiments, the combination of the record management system 104 and the consolidated database 120 can include electronic medical records (EMR) and/or electronic health records (EHR) and software to manage and access the EMR and/or the EHR. When storing, accessing, sharing, sensitive or protected data, the record management system 104 and the consolidated database 120 can be configured to protect the information. For example, the record management system 104 and the consolidated database 120 can be configured to de-identify the data, encrypt the data, or a combination thereof. The consolidated database 120 may be a proprietary or specialized system that has targeted functionality depending on the needs of the user/industry. For example, traditional EMR and/or EHR systems are frequently limited on their ability to aggregate information from multiple data sources, such as the client database 118, the service provider database 108, and the third-party database 122, etc., and visualize the data in a meaningful way.

In some embodiments, the record management system 104 can include an eligibility engine 114. The eligibility engine 114 can be configured to collect, aggregate, or otherwise access information from the consolidated database 120 and/or from a plurality of data sources. For example, the eligibility engine 114 can access data from the client database 118, the third-party database 122, and/or the service provider database 108. The data from different data sources can be combined in a manner for use by the eligibility engine 114, for example, as described in more detail in provisional patent application 63/387,859, which is incorporated by reference herein in its entirety. In some embodiments, the eligibility engine 114 can compare candidate record data from one or more data sources against trial requirement data. The trial requirement data can include any combination of data specifying eligibility requirements for one or more trials. For example, the trial requirement data can include inclusion and exclusion criteria for the one or more trials. The eligibility engine 114 can aggregate, organize, and compare data from all data sources when making determinations whether a candidate(s) is eligible for a trial(s).

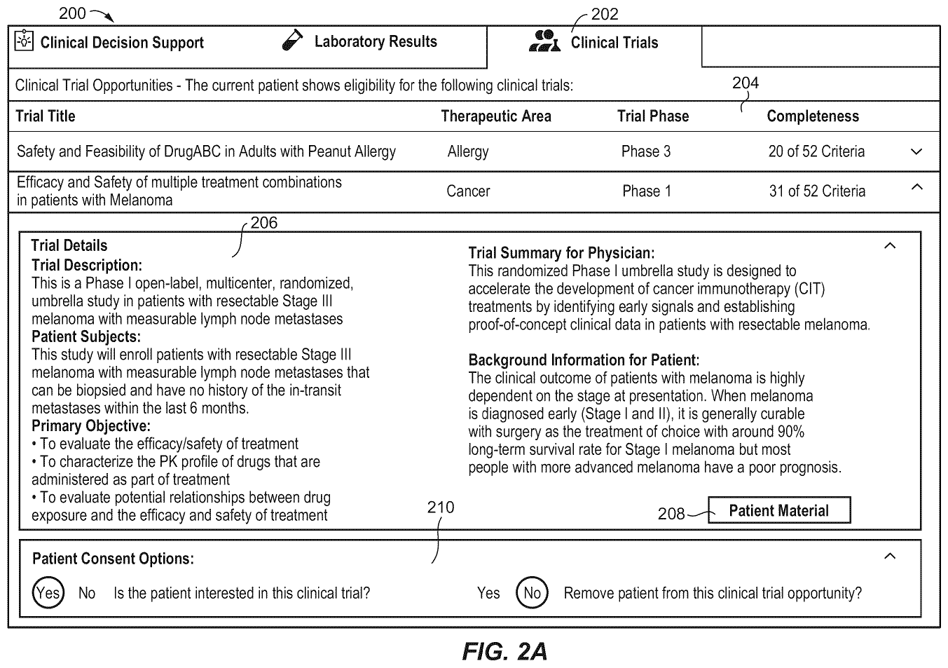

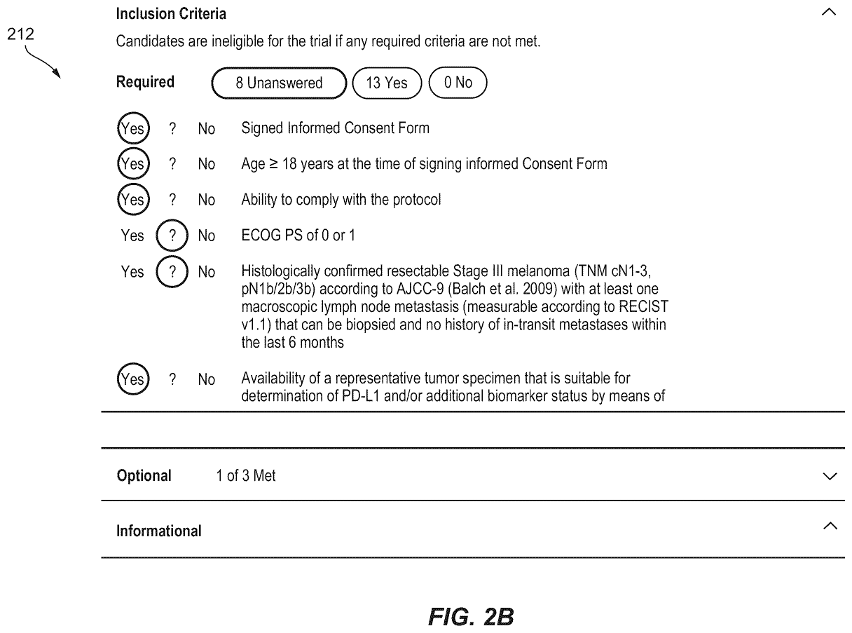

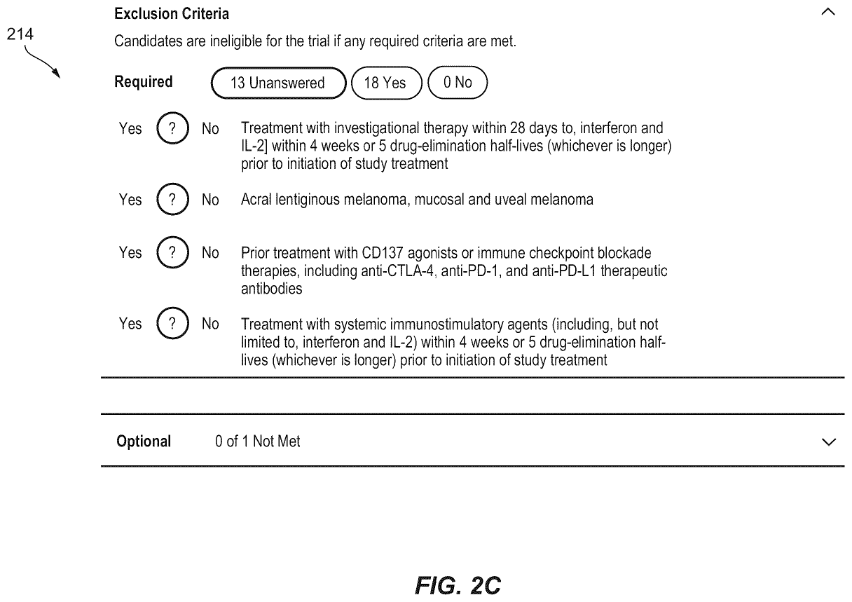

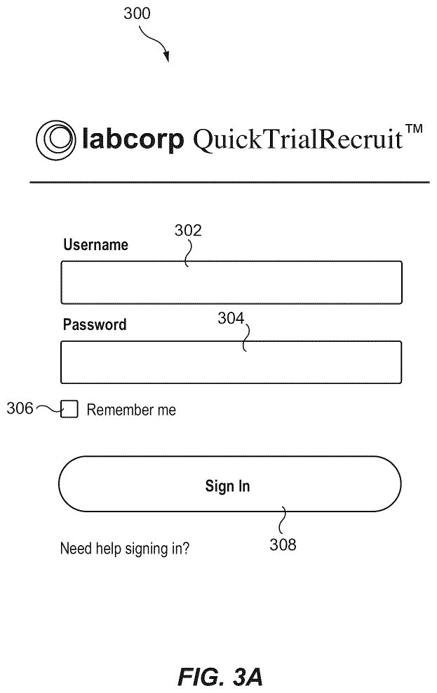

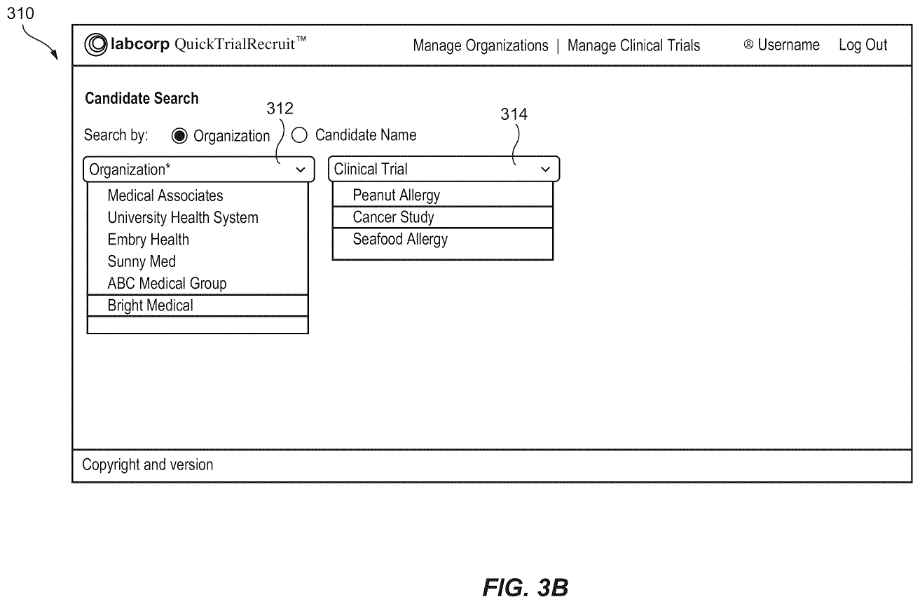

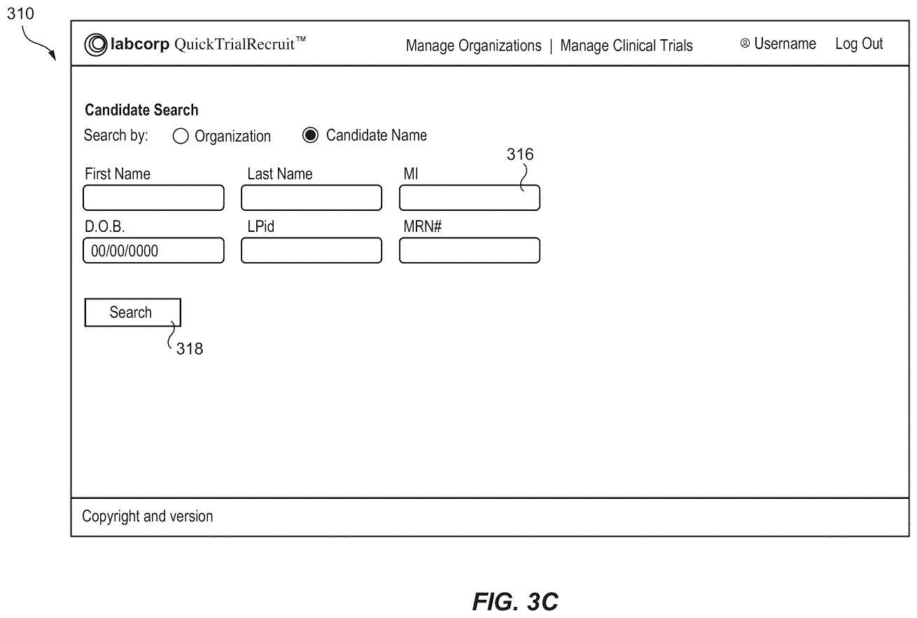

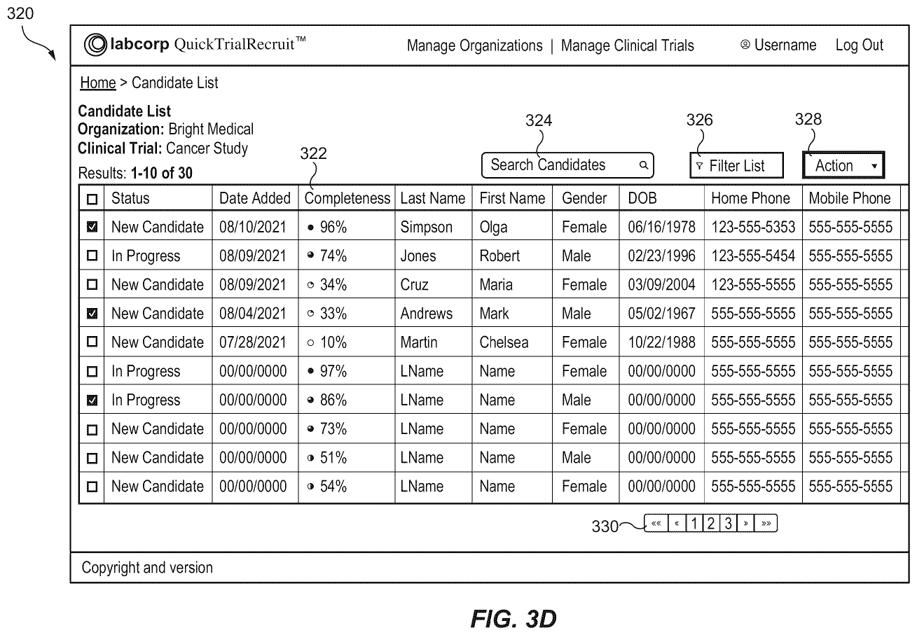

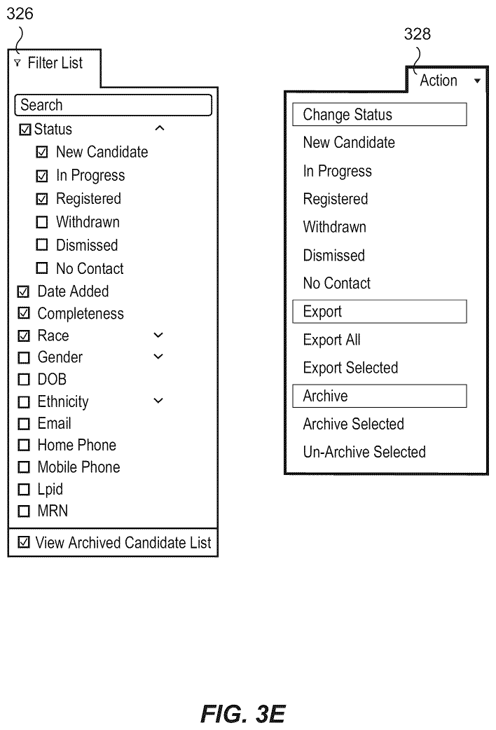

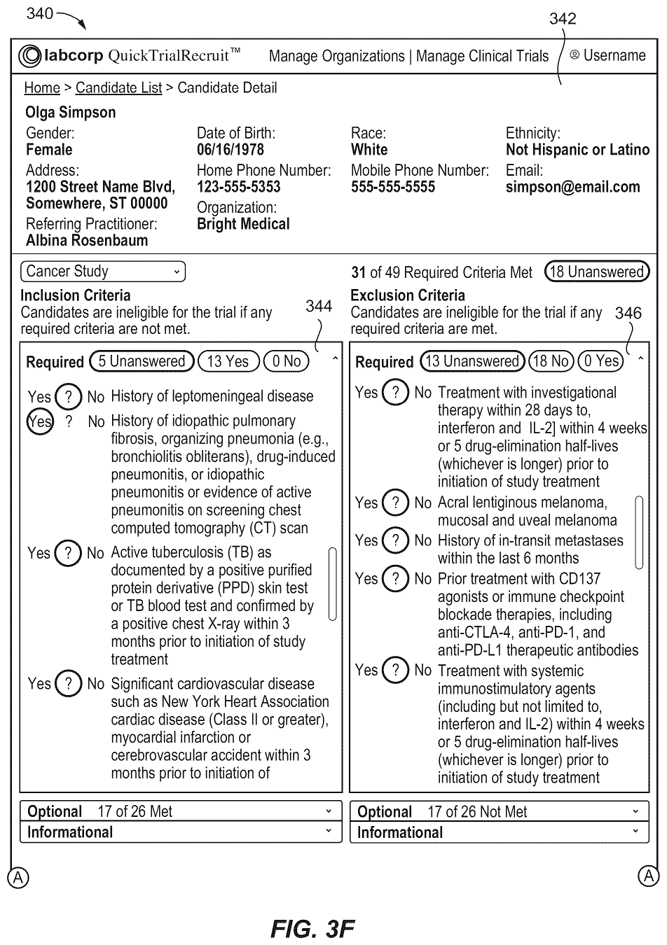

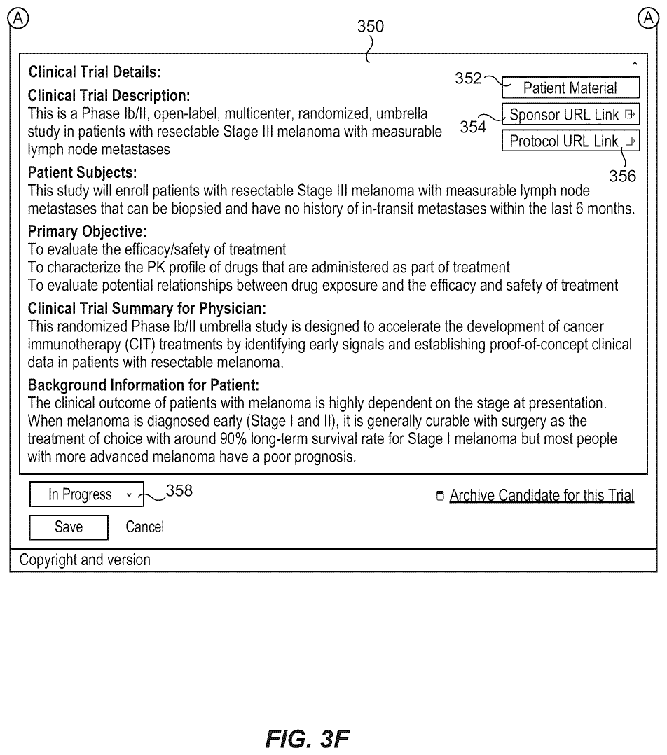

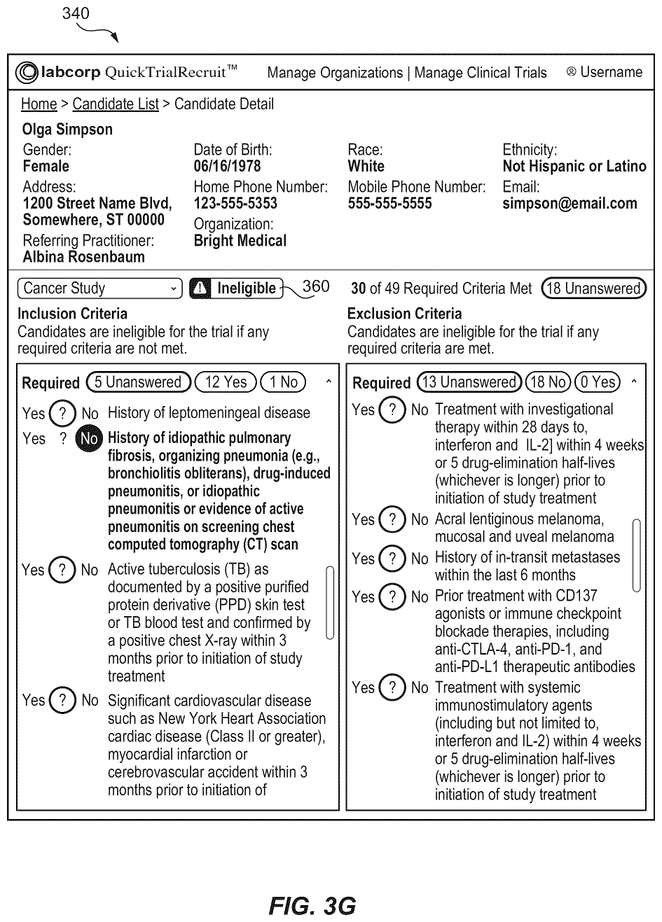

Referring to FIG. 1B, a block diagram illustrating some of the functional components for providing the trial eligibility processing, for example, by the eligibility engine 114 of FIG. 1A, is provided. In some embodiments, the eligibility architecture can include three layers: a presentation layer 150, application layer 155 and database layer 160. The presentation layer 150 can include a set of graphical user interfaces 165, 170 through which users, such as practitioners or administrators, review information (e.g., medical information, history, etc.) and eligibility determinations for candidate(s) and/or trials. The graphical user interfaces 165, 170 can include any combination of graphical user interfaces in accordance with the present disclosure, including but not limited to, trials tab 202 or a trial candidate module as described with respect to FIGS. 2A-3G. The graphical user interfaces 165, 170 can be displayed on one or more workstations or on one or more personal computers. In general, graphical user interfaces 165, 170 can be displayed on any computational system, and although two GUIs are shown in FIG. 1B it should be understood that any number of GUIs can be developed and provided in accordance with the aspects described herein.

In some embodiments, the graphical user interfaces 165, 170 can be coupled to one or more application servers within application layer 155. Application servers 175 and 180 implement the operations to facilitate eligibility determinations from a plurality of data sources (e.g., within the consolidated database 120), in doing so they communicate and process information between graphical user interfaces 165, 170 and the communication network. In various embodiments, the application servers 175 and 180 facilitate the eligibility determinations of data aggregated from a plurality of data sources through a set of mechanisms described herein. Application servers 175 and 180 may be located at a number of locations in a distributed computing system, including at a computational server or a database server, and may be in communication with any of the user interfaces in the presentation layer.

In some embodiments, the application servers 175 and 180 can be coupled to a database management system 185 (e.g., eligibility engine 114 and/or record management system 104 as discussed with respect to FIG. 1A) within the database layer 160. Database management system 185 can be any suitable type of custom-made or commercially available database system for managing storage and retrieval of data. In some embodiments, database management system 185 is configured to collect, aggregate, and format data from databases 190, 195 (e.g., client database 118, service provider database 108, third-party database 122 with respect to FIG. 1A), and the like. Exemplary database servers include without limitation those commercially available from Oracle, Microsoft, Sybase, IBM and the like. Database management system 185 can include or otherwise be communicatively coupled with databases 190, 195 (e.g., record management system 104 and record management system 116 as described with respect to FIG. 1A). The data received from each of the databases 190, 195 can be stored formatted in a common format such that common data can be aggregated together and used by the eligibility engine 114. For example, all data for Jane Doe can be aggregated into a data structure, deduplicated, and tagged with a data source.

Returning to FIG. 1A, in some embodiments, the eligibility engine 114 can be configured to evaluate candidate records against a plurality of trial criteria to determine if a candidate and/or what candidates are eligible or potentially eligible for a trial. The evaluation can be performed by comparing one or more characteristics, questions/answers, test results, etc. for a candidate against one or more criteria for a trial. The evaluation can be performed using any combination of methods. For example, the evaluation may include a comparison which can be performed using a matching algorithm, artificial intelligence, machine learning, neural network, etc. or a combination thereof. An eligibility of the candidate can be based on a score, ranking, confidence score, etc. of the determination, and, in addition or alternatively, may be based on a professional recommendation. The eligibility engine 114 can be configured to access an aggregation of candidate records and trial records when making a determination.

In some embodiments, the eligibility engine 114 can aggregate medical data from disjunctive sources. The disjunctive sources can include a combination of EHR systems, EMR systems, specialized facilities (e.g., testing facilities), and any other suitable disjunctive sources. Other systems, such as EHR systems or EMR systems, may not have access to data from each of the disjunctive sources. For example, a trial coordinator, such as a medical professional and/or clinical research coordinator, may be able to access data relating to a candidate from an EHR system or EMR system; but may not be able to access data relating to the candidate from a specialized facility or other related testing facilities. Thus, in conventional systems, to access the data relating to the candidate from the specialized facility or other related testing facilities, the trial coordinator or the candidate may submit a records request to the specialized facility or other related testing facilities to receive the relevant data. However, the records request may take an extended period of time over hours, days, or weeks to be granted. Additionally, during the extended period of time, medical conditions or treatments with respect to the candidate may change. Accordingly, without access to the most recent and relevant data relating to the candidate from the disjunctive sources, the trial coordinator may not be able to assess an eligibility of the candidate for a trial in a timely fashion.

Determining eligibility for the candidate to participate in the particular trial can be difficult. For example, the particular trial may involve various variables and restrictions that limit an available population of candidates that are eligible to participate in the trial. Without data from the disjunctive sources, the trial coordinator may not be able to make an accurate judgement regarding whether the candidate is eligible for the particular trial.

The eligibility engine 114 can address the above-described difficulties and additional difficulties. For example, the eligibility engine can retrieve data for a candidate from various disjunctive sources. In one such example, the eligibility engine can communicate with EMR systems or EHR systems, a specialized facility, and any other suitable entity that generates or stores data relating to the candidate. The eligibility engine 114 can request and receive data from the disjunctive sources, can de-duplicate the received data, and can provide assistance to a trial coordinator in determining and evaluating eligibility for the candidate in a trial. Additionally, the eligibility engine 114 can provide obscure or otherwise difficult-to-find data that can help determine the eligibility of the candidate for trials.

The eligibility engine 114 can be configured to generate customizable graphical representations illustrating information about a trial and how a candidate is a match, is not a match, or potentially is a match to be eligible for the trial. For example, the eligibility engine 114 can generate a graphical user interface (GUI) that can include one or more displays showing an individual data set, an aggregation of historical data sets, a collection of aggregated historical data sets, etc. The data sets can include any combination of data. For example, the data sets can be related to medical data, test data, etc. which can include test results, biometric data, etc. The data sets can also include supplemental data that correspond to the data sets. The graphical representations can also include prompts to be delivered to solicit additional information about the candidate to make further determinations about candidate eligibility. For example, the eligibility engine 114 can generate a GUI containing one or more displays providing prompts to the trial coordinator to ask questions to the candidate, receiving feedback from the candidate, receiving election(s)/rejection(s) from the candidate for participation within the trial, etc. The eligibility engine 114 can be configured to review, interpret, analyze, modify and/or determine how to best visualize the data. In some embodiments, the eligibility engine 114 can be configured to aggregate criteria for trials.

In some examples, the eligibility engine 114 may be configured to retrieve information about a trial and how a candidate is a match substantially in real-time upon request, or automatically upon generation of the GUI. The information may include a collection of candidate records from the various disjunctive sources and provide a user (e.g., trial coordinator, authorized user, medical professional, etc.) access at an onsite facility or remote location so that the user may use the GUI, which has organized the aggregated information from the various disjunctive sources, to update various criteria (e.g., inclusion and/or exclusion criteria as discussed in more detail in FIGS. 2B, 2C, and 2F). The eligibility engine 114 may display, using the GUI, some or all information about a candidate that is a match, is not a match, or potentially is a match to be eligible for the trial. In addition, the eligibility engine 114 may convert, shorten, or display differently, one or more portions (e.g., information determined to be important to the trial) of the GUI to more clearly, concisely, and cleanly display aggregated information from the various disjunctive sources (e.g., trial details graphical element 206 of FIG. 2A). For example, the eligibility engine 114 may select one or more bullets, summaries, concepts, or similar for displaying the information about the trial and/or candidates.

In some examples, the eligibility engine 114 may store the updated criteria (e.g., inclusion and/or exclusion criteria as discussed in more detail in FIGS. 2B, 2C, and 2F) in a standardized format in a centralized hub (e.g., cloud server) for ease of retrieval. By way of a non-limiting example, a trial coordinator may receive information from a candidate regarding age of consent, ability to comply with the protocol, and similar information and store the updated criteria on a facility specific network for later retrieval. In addition, or alternatively, the eligibility engine 114 may automatically generate a notification and/or message (e.g., a GUI pop-up, email, etc.) containing confirmation of storing the updated criteria. The eligibility engine 114 may continuously monitor for updates from the trial coordinator, or may monitor for updates according to a pre-defined schedule, frequency, or by request. In addition, or alternatively, the eligibility engine 114 may notify any suitable number of candidates or trial coordinators when the updated criteria is missing information (e.g., unanswered questions) and update the GUI accordingly to display only sections which are missing information. In some examples, the eligibility engine 114 messages some or all trial coordinators and/or candidates when new criteria is added, information is updated, and/or conditions require candidate input (e.g., informing candidates of government and/or institutional policy changes related to the trial).

Each of the components within the system 100 can include a plurality of devices configured to communicate with one another over a communication network 112. In some embodiments, the communication network 112 can include multiple communication networks. In some embodiments, the client system 102, the record management system 104, and the service provider system 106 can be configured to establish a connection and communicate over communication network 112 to carry out aspects of the present disclosure. As would be appreciated by one skilled in the art, the communication network 112 can include any combination of known networks. For example, the communication network 112 may be a combination of a mobile network, WAN, LAN, or other type of network. The communication network 112 can be used to exchange data between the client system 102, the record management system 104, the service provider system 106, and/or to collect data from additional sources.

Continuing with FIG. 1A, the combination of hardware and software that make up the system 100 are specifically configured to provide a technical solution to a particular problem utilizing an unconventional combination of steps/operations to carry out aspects of the present disclosure. In particular, the system 100 can execute a unique combination of steps to aggregate, organize, format and visualize information provided to a user in an advantageous manner.

FIG. 2A is a non-limiting clinical example of a first portion 200, which may be or include a top portion, of a trials tab 202 for assisting a physician in identifying possible trial opportunities for a candidate during a candidate encounter in accordance with various embodiments. Graphical elements included in the ‘Trials’ tab 202 can include a list 204 of trial opportunities. Each trial opportunity in the list 204 can be characterized by columns with column titles including ‘trial title’, ‘therapeutic area’, ‘trial phase’, and/or ‘completeness’. Examples of categories for the ‘therapeutic area’ column can include Cancer, Allergy, Respiratory, Neurologic Disease, Dermatology, Immunology, Cardiovascular, Cell and Gene therapy, Ophthalmology, Gastrointestinal, Endocrine, Urologic, Autoimmune, Infectious Disease, Obesity, Hematologic. Rare Genetic Disease, Renal, HIV/AIDS, etc. Entries in the ‘Completeness’ column can specify a number of criteria associated with the trial met by the candidate. Entries for the ‘Trial Phase’ column can include Phase 1, Phase 2, Phase 3, or Phase 4, etc.

The list 204 of trial opportunities can be arranged in any order according to entries in any of the columns. For example, the list 204 can be arranged in alphabetical order or reverse alphabetical order according to entries in the ‘Trial Title’ or ‘Therapeutic Area’ columns. The list 204 can be arranged according to entries in the ‘Trial Phase’ column, from trials in an earliest phase, such as Phase 1, to trials in a latest stage, such as Phase 4, or vice versa. The list 204 can be arranged according to entries in the ‘Completeness’ column as well from most complete to least complete or vice versa. An entry that is considered most complete can be based on a highest number of criteria met or on a highest percentage of criteria met. In some examples, the physician can control an arrangement of the list 204 by tapping (or double tapping) on a column name. For example, tapping on the ‘Therapeutic Area’ column heading once can arrange the list 204 in alphabetic order by ‘Therapeutic Area’ entry. Double tapping on the ‘Therapeutic Area’ column heading can arrange the list 204 in reverse alphabetic order by ‘Therapeutic Area’ entry.

The physician can select a specific trial opportunity from the list 204 by tapping on any location in a row for the specific trial within the list 204. Once the specific trial opportunity is selected, a trial details graphical element 206 can be revealed within the trials tab 202. The ‘Trial Details’ graphical element 306 can appear within the trials tab 202 under the list 204 of trial opportunities as shown in FIG. 2A. The trial details graphical element 206 can include information to assist the physician to learn about the trial opportunity and help the physician explain the trial opportunity to the candidate. The information included within the trial details graphical element 206 can include a trial description, a patient subject section, a primary objective(s), a trial summary for the physician, background information for the candidate, and the like. The trial details graphical element 206 can also include a candidate material button 208. When the candidate material button 208 is selected, additional details regarding the trial opportunity can be printed out and provided to the candidate so that the candidate can take additional time to consider whether or not the candidate is interested in further pursuing the trial opportunity.

The trials tab 202 can also include a graphical element 210 associated with candidate consent options. The graphical element 210 associated with candidate consent options can appear at a bottom of the first portion 200. Within the graphical element 210, the physician can be provided an opportunity to answer questions. The questions can be related to candidate consent. Examples of the questions can include: “Is the candidate interested in this trial?” and “Remove candidate from this trial opportunity?”. The physician can answer all of the questions, a portion of the questions, or none of the questions. In some examples, an answer to one or multiple questions can automatically provide an answer to other questions. For example, an answer of ‘yes’ to the “Is the candidate interested in this trial?” question can automatically provide an answer of ‘no’ to the “Remove candidate from this trial opportunity?” question. But note that an answer of ‘no’ to the “Is the candidate interested in this trial?” does not necessarily provide an answer of ‘yes’ to the “Remove candidate from this trial opportunity?” question. When the answer to the “Is the candidate interested in this trial?” question is ‘yes’, the candidate can be added to a candidate list associated with the trial opportunity.

FIG. 2B is an example of a second portion 212, which may be or include a lower-left portion, of a trials tab of a GUI for assisting a physician in identifying possible trial opportunities for a candidate during a candidate encounter in accordance with various embodiments. The second portion 212 can include a summary of inclusion criteria for a selected trial opportunity. If a candidate fails to meet even a single criterion within the inclusion criteria, the candidate can be excluded from the trial opportunity. The summary of inclusion criteria can be presented as a list of yes or no questions. Each question in the list of yes or no questions can be characterized as ‘yes’, ‘no’, or ‘unanswered’. Referring to FIG. 2A, the list 204 of trial opportunities can exclude any trial opportunity that includes at least one ‘no’ in the list of yes or no questions of the summary of inclusion criteria.

The physician can assist with the list of yes or no questions of the summary of inclusion criteria. The physician can provide answers to unanswered questions, confirm or correct answered questions, or provide any of the yes or no questions to the candidate. Each question displayed in the lower left portion 212 can include a yes option, a ‘?’ option, or a ‘no’ option. Unanswered questions can be characterized with ‘?’ entries by default. The physician can change an answer to any of the yes or no questions by tapping on the yes option, the ‘?’ option, or the ‘no’ option for the yes or no question. There may be some yes or no questions that the physician is unable to answer. Answers to the yes or no questions in the second portion 212 can assist the physician in completing the graphical element 210 associated with candidate consent options displayed in FIG. 2A. For example, if, after reviewing all the yes or no questions of the summary of inclusion criteria, answers only include yes or ‘?’ answers; the physician can ask the candidate if the candidate is interested in the trial. If any of the answers is a ‘no’, the physician can select ‘no’ for the “Is the candidate interested in the trial?′ question and ‘yes’ to the ‘Remove candidate from the trial opportunity?” question. In some examples, a no answer to any of the yes or no questions in the second portion 212 of the trials tab 202 can automatically cause the answer to the ‘Remove candidate from the trial opportunity?” question to become ‘no’.

FIG. 2C is an example of a third portion 214, which may be or include a lower-right portion, of a trials tab of a GUI for assisting a physician in identifying possible trial opportunities for a candidate during a candidate encounter in accordance with various embodiments. The third portion 214 can include a summary of exclusion criteria for a selected trial opportunity. If a candidate meets even a single criterion within the exclusion criteria, the candidate can be excluded from the trial opportunity. The summary of exclusion criteria can be presented as a list of yes or no questions. Each question in the list of yes or no questions can be characterized as ‘yes’, ‘no’, or ‘unanswered’. Referring to FIG. 2A, the list 204 of trial opportunities can exclude any trial opportunity that includes at least one ‘yes’ in the list of yes or no questions of the summary of exclusion criteria.

The physician can assist with the list of yes or no questions of the summary of exclusion criteria. The physician can provide answers to unanswered questions, confirm or correct answered questions, or provide any of the yes or no questions to the candidate. Each question displayed in the lower right portion 214 can include a yes option, a ‘?’ option, or a ‘no’ option. Unanswered questions can be characterized with ‘?’ entries by default. The physician can change an answer to any of the yes or no questions by tapping on the yes option, the ‘?’ option, or the ‘no’ option for the yes or no question. There may be some yes or no questions that the physician is unable to answer. Answers to the yes or no questions in the third portion 214 can assist the physician in completing the graphical element 210 associated with candidate consent options displayed in FIG. 2A. For example, if, after reviewing all the yes or no questions of the summary of exclusion criteria, answers only include no or ‘?’ answers; the physician can ask the candidate if the candidate is interested in the trial. If any of the answers is a ‘yes’, the physician can select ‘no’ for the “Is the candidate interested in the trial?” question and ‘yes’ to the ‘Remove candidate from the trial opportunity?” question. In some examples, a yes answer to any of the yes or no questions in the third portion 214 can automatically cause the answer to the ‘Remove candidate from the trial opportunity?” question to become ‘yes’.

FIG. 2A-2C each depict one or more portions of a non-limiting GUI. It should be understood that while this non-limiting example depicts a clinical trial interface, any suitable a GUI for different types of trials may be implemented according to embodiments herein. For example, some different types of trials may include long term statistical trials, short term statistical trials, population trials, random sampling trials, social science trials, environmental trials, educational trials, case study trials, longitudinal study trials, correlation trials, or combinations thereof.

FIG. 3A is an example of a login GUI 300 for a trial candidate GUI to assist authorized users with identifying candidates in trials in accordance with various embodiments. The login GUI 300 can help ensure that only authorized users can access information provided by the trial candidate module. The authorized users can include researchers, trial coordinators, investigators, principal investigators, clinical research associates, clinical researchers, etc.

The login GUI 300 can include a username field 302, a password field 304, a ‘Remember me’ option 306, a sign-in button 308, etc. An authorized user can login by entering a username in the username field 302, a password in the password field 304, and clicking the sign-in button 308. In some examples, clicking the sign-in button 308 can trigger two-factor authorization (2FA). 2FA can involve sending a verification text message to a mobile device associated with the authorized user or sending a verification email to the authorized user. The authorized user can choose to check the ‘Remember me’ option 306. When the ‘Remember me’ option 306 is checked, the login for the authorized user can be remembered for a duration of time. The duration of time can include multiple hours, a 24-hour period, multiple days, etc. During the duration of time, each time the authorized user reopens the trial candidate module, the authorized user will automatically be logged in. Logging into the trial candidate module will open a Candidate Search GUI, which is described in more detail in FIGS. 3B and 3C.

FIG. 3B is an example of a candidate search GUI 310 in organization search mode for a trial candidate module to assist authorized users with identifying candidates in trials in accordance with various embodiments. Once logged in to the trial candidate mode, an authorized user gains access to the candidate search GUI 310. The authorized user can select one of two search modes for the candidate search GUI 310. The two search modes include an organization search mode and a candidate name search mode. FIG. 3B depicts the organization search mode for the candidate search GUI 310.

The organization search mode for the candidate search GUI 310 can include two drop down menus. One of the two drop-down menus can be an organization drop-down menu 312. The organization drop-down menu 312 can allow the authorized user to select an organization from a group of organizations. In FIG. 3B, six organizations are depicted in the group of organizations. Examples of organizations can include Medical Associates, University Health System, Embry Health, Sunny Med, ABC Medical Group, Bright Medical, etc. FIG. 3B depicts an authorized user selection of the Bright Medical organization. The group of organizations can include any number of organizations including one organization. While this example presents a number of medical organizations, it should be understood that any number of organizations outside of the medical field may be populated if relevant to a particular trial. For example, non-medical organizations may include universities, political organizations, governments, commercial entities such as restaurants, retail stores, data centers, etc., or combinations thereof.

Another of the two drop down menus can be a trial drop-down menu 314. The trial drop-down menu 314 can provide a list of trials associated with a selected organization from the organization drop-down menu 312. The authorized user can select a trial from the trial drop-down menu 314. In some examples, the authorized user can select multiple trials from the trial drop-down menu 314. FIG. 3B depicts three trials associated with the selected Bright Medical organization. Examples of the trials can include a peanut allergy trial, a cancer study trial, a seafood allergy trial, etc. Each selected organization can include any number of trials including one trial or zero trials. While this example presents a number of medical trials, it should be understood that any number of trials outside of the medical field may be populated if relevant to a particular organization.

After selecting an organization from the organization drop-down menu 312 and a trial associated with the selected organization from the trial drop-down menu 314, the authorized user can click on a search button. In FIG. 3B, the search button is hidden behind the organization drop-down menu 312, but may be located at any suitable location. Clicking on the search button can open a Candidate List GUI, which is described in more detail in FIG. 3D.

FIG. 3C is an example of a candidate search GUI 310 in candidate name search mode for a trial candidate module to assist authorized users with identifying candidates in trials in accordance with various embodiments. Once logged in to the trial candidate mode, an authorized user gains access to the candidate search GUI 310. The authorized user can select one of two search modes for the candidate search GUI 310. The two search modes include an organization search mode and a candidate name search mode. FIG. 3C depicts the candidate name search mode for the candidate search GUI 310.

The candidate name search mode for the candidate search GUI 310 can contain multiple fields 316 for providing identifying information associated with a particular candidate in a trial. Six fields 316 are shown in FIG. 3C, though only one is associated with a numerical indicator in FIG. 3C. Examples of the fields can include a first name field, a last name field, a middle initial (MI) field, a date of birth field, a medical record number (MRN) field, other field associated with identifying information, etc. Examples of other fields associated with identifying information can include candidate identification number fields, a passport number fields, driver's license number fields, and the like. To search for the particular candidate, the authorized user can provide entries in just one of the fields 316, all of the fields 316, or any number of the fields 316. Some of the fields 316 can be left blank by the authorized user.

After providing entries in the fields 316, the authorized user can click on a search button 318. Clicking on the search button 318 can open a Candidate Detail GUI. The Candidate Detail GUI is described in more detail in FIG. 3G.

FIG. 3D is an example of a candidate list GUI 320 for a trial candidate module to assist authorized users with identifying candidates in trials in accordance with various embodiments. The candidate list GUI 320 can provide information associated with candidates for a trial associated with an organization. As noted above, the candidate list GUI 320 can be opened when an authorized user performs a search on the candidate search GUI 310 in organization search mode. The candidate list GUI 320 can include several features to assist authorized users in assessing potential candidates in trials. The features can include a table of candidates 322, a search candidates field 324, a filter feature 326, an action feature 328, buttons 330 for toggling between pages of candidate lists, etc.

The table of candidates 322 can include any number of rows and any number of columns. The table of candidates 322 can list all candidates being considered for participation in the trial associated with the organization. The table of candidates 322 depicted in FIG. 3D includes ten columns and 11 rows, including a row for column headings. Examples of column headings can include a status heading, a date added heading, a completeness heading, a last name heading, a first name heading, a gender heading, a date of birth heading, a home phone heading, a mobile phone heading, etc. A selection column can also be included in the table of candidates 322 with a heading that includes an unfilled square icon.

Examples of entries in the status column can include ‘new candidate’, ‘in progress’, ‘withdrawn’, ‘dismissed’, ‘no contact’, etc. When a candidate is first added to the list of all candidates being considered for participation, the status column entry for the candidate can automatically be set to the ‘new candidate’ entry. Physicians can be granted an additional opportunity to communicate a consent of a candidate within the trials tab in addition to conventional means of communicating consent, such as communicating consent directly to investigators associated with the trial. Candidates can be added to the list when a physician submits a yes answer to a question “Is the candidate interested in the trial?”. For example, the physician can provide the yes answer to this question within a graphical element 210 associated with candidate consent options within a ‘Trials’ tab such as within the ‘Trials’ tab 202 described in FIG. 2A. An available candidate that expressed interest in a similar trial but was disqualified from the similar trial can automatically be added to the list of all candidates being considered for participation. The authorized users can also add new candidates to the list. After a predetermined duration of time passes after the candidate is added to the list, the status column entry can automatically be changed from ‘new candidate’ to ‘in progress’. For example, the predetermined duration of time can include a set number of hours, days, weeks, months, etc. An entry in the status column of ‘no contact’ can indicate that the candidate does not wish to be contacted regarding the potential trial or any potential trial.

Entries in a column with the completeness column heading can include a completion score presented as a percentage value with a partially filled pie icon that can help an authorized user visualize the percentage value. The percentage value can represent a percentage of inclusion criteria questions answered, a percentage of exclusion criteria answered, or a percentage of the combination of both types of criteria questions. For example, if 14 of 50 inclusion criteria questions have been answered, and if 23 of 50 exclusion questions have been answered, then the percentage value entry in the completeness column can be 37% (37 answered questions of 100 combined questions).

The authorized user can use the selection column in several ways. Each candidate row can include an empty square by default in the selection column. The authorized user can click on the empty square and produce a check mark in the empty square to indicate that the candidate row has been selected. Selecting the candidate row can lead to an additional action. For example, the candidate row can be selected to open a candidate detail GUI associated with the candidate. The candidate detail GUI will be described in more detail in FIG. 3F. The authorized user can perform other actions to a selected candidate row, such as choosing the candidate to be a candidate in the trial or deleting the candidate from the trial candidate list. Multiple candidate rows can be selected so that a single action can be performed on each candidate row simultaneously.

If a name of a candidate is unknown, an entry in the first name column for the unknown candidate can be set to a default word or token, such as Name. The entry in the last name column for the unknown candidate can be set to a different default word or token, such as LName. Unknown dates, such as an unknown date of birth can take on a default value as well, like 00/00/0000.

The table of candidates 322 can include multiple pages. For example, a first page of the table of candidates 322 can include 11 rows, one row per candidate plus a top row for the column headers. If there are more than ten candidates for the trial, additional candidates can be included in rows on a different page of the table of candidates 322. The buttons 330 for toggling between pages of candidate lists can allow the authorized user to view the additional candidates on other pages.

An order of the list described by the table of candidates 322 can be rearranged when the authorized user clicks (or double-clicks) on one of the column headers. For example, the authorized user can click on the column with the date of birth heading and the list will be reordered based on the date of birth for the candidates. For example, the list can be reordered starting with a youngest candidate in a top row and the oldest candidate in the bottom row. In another example, the authorized user can double-click on the column with the date of birth heading and the candidates can be reordered, from oldest candidates at the top to youngest candidates at the bottom of the table of candidates 322. In some examples, candidates with unknown date of births can be placed in the top rows within the reordering of the list. In other examples, the candidates with unknown date of births can be placed in the bottom rows.

The authorized user can use the search candidates field 324 to locate a row associated with a specific candidate from among all of the rows in the table of candidates. The authorized user can enter a first name, last name, date of entry, a date that a candidate was added to the list, home phone number, a gender, or mobile phone entry. In some examples, the authorized user can type an entry in the search candidates field 324, press enter on a keyboard, and the table of candidates 322 of the candidate list GUI 320 will reset so that only a row (or rows) categorized by the entry in the search candidates field 324 will be displayed. For example, the authorize user can type the word ‘John’ in the search candidates field 324, press enter, and the table of candidates 322 will only display rows that include ‘John’ in at least one column of the row. The rows can include all candidates with the first name John and all candidates that include ‘John’ in at least a portion of the last name, such as ‘John’ (like Elton John), ‘Johnson’ or ‘Johnathon’.

The authorized user can also use the filter feature 326 and the actions feature 328. These two features can be used to further organize the table of candidates 322 and perform activities associated with selection candidates in the trial. The filter feature 326 and the actions feature 328 will be described in more detail in FIG. 3E.

FIG. 3E are examples of a filter feature 326 and an actions feature 328 included in a candidate list GUI for a trial candidate module to assist authorized users with identifying candidates in trials in accordance with various embodiments. The filter feature 326 can include a drop-down menu that can include elements that allow an authorized user to filter results displayed in a table of candidates, such as the table of candidates 322 described in FIG. 3D. The elements in the drop-down menu of the filter feature can coincide with column headings of the table of candidates. Examples of the elements include status, date added, completeness, race, ethnicity, gender, date of birth, email, home phone, mobile phone, medical record number, any other identifying number, etc. Selecting one element can arrange rows in the table of candidates 322 based on an order of the entries in a column associated with the one element. For example, if only the ‘date added’ element is selected, the list can arrange the rows based on date added from earliest added to latest added or vice versa.

Each element in the filter feature 326 can include a secondary drop-down menu. If the element is associated with a column heading of the table of candidates 322, then the secondary drop-down menu can include all possible entries within the associated column. For example, the status element can include a secondary drop-down menu that includes all possible entries in a status column of the table of candidates, such as new candidate, in progress, registered, withdrawn, dismissed, no contact, etc.

In some embodiments, the action feature 328 can be used in conjunction with a selection column of the table of candidates 322. The selection column can allow the authorized user to select a single row, several rows, or a group of rows within the table of candidates 322. If several rows are selected, the several rows can include selected rows on multiple pages of the table of candidates. The authorized user can use the selection column to determine which rows to perform an action upon. Each row can be associated with a single candidate for a trial.

The action feature 328 can include a drop-down menu with possible actions to perform to rows of the table of candidates. For example, the authorized user can use the action feature to change an entry in the status column for selected rows. The authorized user can use the action feature 328 to export all rows or just selected rows into a database associated with a trial. When a row is exported into the database, a candidate associated with the row can be considered a candidate in the trial. The action feature can also be used to archive or un-archive a row. Archiving a row can temporarily remove the row from the table of candidates until the row is un-archived.

FIG. 3F is an example of a candidate detail GUI 340 for a trial candidate module to assist authorized users with identifying candidates in trials in accordance with various embodiments. The candidate detail GUI 340 can include several sections, such as biographical info section 342, an inclusion criteria section 344, an exclusion criteria section 346, and a trials details section 350. The biographical info section 342 can include characteristics of a candidate for a trial including gender, date of birth, race, ethnicity, referring practitioner, etc. The biographical info section 342 can also include contact information for the candidate such as email, home phone, mobile phone, address, etc.

The inclusion criteria section 344 and the exclusion criteria section 346 can both include a set of yes or no questions that can assist in determining if the candidate qualifies for a trial. The set of yes or no questions can include answered and unanswered questions. The authorized user can browse the questions, provide answers to unanswered questions, or confirm answers to questions in both the inclusion criteria section 344 and the exclusion criteria section 346. A drop-down menu above a heading for the inclusion criteria section 344 can allow the authorized user to select a different trial that the candidate can potentially enter.

The trials details section 350 can provide the authorized user with additional information associated with a selected trial. The additional information can include a description of the trial, a description describing candidates that can participate in the trial, objectives for the trial, a trial summary for a physician, and background information for a candidate. The trial details section 350 can include several buttons for the authorized user. The buttons can include a candidate material button 352, a link button 354 to a website for a trial sponsor, a link button 356 to a website with protocols for the trial, and the like. The candidate detail GUI 340 can also include a drop-down menu 358 for changing a status of the candidate. When the authorized user selects a status from the drop-down menu 358, the authorized user can save the change to the status or cancel the change.

FIG. 3G is an additional example of a candidate detail GUI 340 for a trial candidate module to assist authorized users with identifying candidates in trials in accordance with various embodiments. The candidate detail GUI 340 can include an indicator 360 that a candidate is ineligible for a selected trial. The indicator 360 can be located next to a trial drop-down menu above an inclusion criteria section in the candidate detail GUI 340 and highlighted in red. Questions in the inclusion criteria section or an exclusion criteria section that indicate that the candidate is ineligible can be highlighted in red as well.

The example GUIs illustrated in FIG. 3B-3G should not be considered limiting. For example, the GUIs may be adjusted to display and/or hide some or all portions (e.g., information displays and/or input regions) of information based on a user and/or coordinator (e.g., medical professional) preference. In some examples, certain GUI sections (e.g., organizations and/or candidate names as in FIG. 3B) may be selectively reorganized based on preference. For example, the organization and candidate name areas may be swapped in place and/or adjusted automatically based on a detected screen space resolution by a processor. The processor may identify each portion to be displayed in some or all GUIs depicted in FIG. 3B-3F and may make a determination on which section to display, where to display the portion, how many portion to display per tab, or combinations thereof. In addition, or alternatively, the processor may automatically determine (e.g., using a machine learning algorithm such as linear regression) which portions to display based on i) portions that are utilized the most by users, ii) portions that are moved the more often by users, iii) portions that are minimized or otherwise hidden by users, iii) portions that are maximized or otherwise expanded by users the most, iv) required portions to be displayed per trial v) portions that require input by the users, or combinations thereof. The adaptability of the trial GUI ensures that each user of the trial GUI may quickly identify portions which need input and/or gather additional information when needed by adjusting the size and/or visibility of the portions of the GUI. The flexibility and adaptability of the trials GUI ensures that a user may enjoy a quick and preference-catered experience without being inundated with excessive information which may not be relevant that the time of use. Additionally, or alternatively, due to the complex nature of trials and candidate selection, reducing and/or reorganizing specific portions of the GUI enables a user to get to most relevant information without having to sift through information which may be redundant and/or unnecessary.

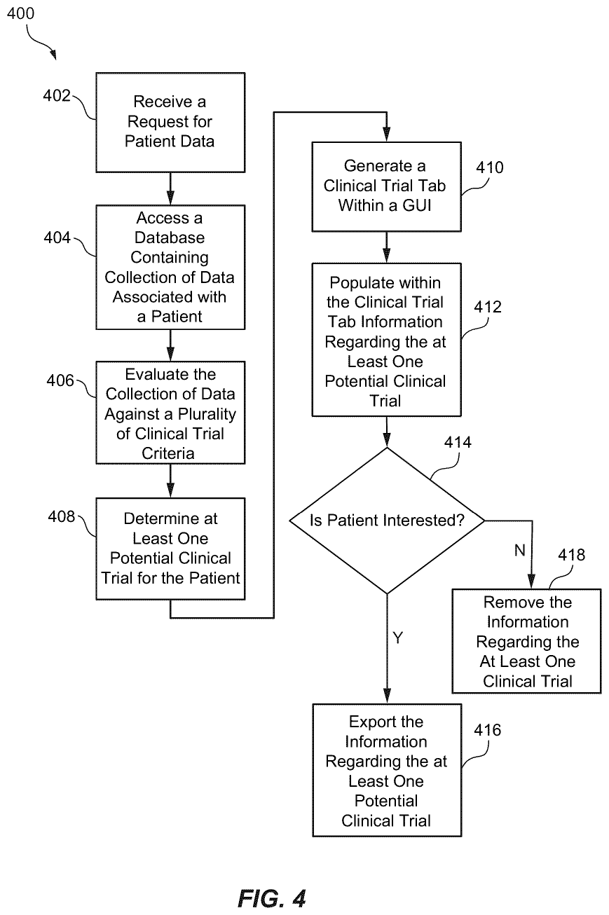

FIG. 4 provides an example flow chart depicting a process 400 in accordance with the aspects of the present disclosure. In particular, FIG. 4 depicts an example of a process 400 for populating and interacting with a graphical user interface including basic information, for example, trials Tab 202, as depicted in FIGS. 2A-2C. The process 400 is discussed with respect to providing trial information (or other types of information) for review by a user (e.g., a healthcare practitioner) and providing criterion related to the trial information, however, the process 400 could be applied to any combination of industries and client/server relationships and is not intended to be limited to the examples provided herein. Initially, the trials dashboard tab of the GUI generates a list of potential trials and information associated with the list from one or more data sources. The generation of the list can be performed in any combination of systems and methods. For example, the generation of the list of potential trials can be provided as described in more detail in provisional patent application 63/387,859, which is incorporated by reference herein in its entirety.

Initially, a user is provided with a GUI depicting one or more options for tests to be visualized. The GUI can be provided by a service provider system 106 through a web browser 110 or application on a client system 102. For example, the user can be provided with the GUI depicting a list of tests, test panels, clinical groups, or a combination thereof. Alternatively, a user can load a visualizer which may have a default starting state. For example, the user can be provided with the GUI without any data populated within the tabs or data lanes.

At step 402, a request for candidate data is received. In some embodiments, the GUI receives an input for the candidate data to be received. For example, the GUI can receive a candidate identification information (or other identifier) for a candidate that the data is to be related to. The candidate identification information (or other identifier) can be obtained through any combination of methods, for example, an input field. The candidate identification information can include any combination of information about the candidate. For example, the candidate identification information can include the name of the candidate, the date of birth of the candidate, the medical record number of the candidate, etc. The GUI can provide data that is specific to the candidate to aid a trial coordinator in providing services to the candidate. The request can be received from a physician for the candidate during a candidate encounter. The physician can be a primary physician or a specialist.

At step 404, a database containing a collection of data associated with the candidate is accessed. In some embodiments, accessing the database can include obtaining all data related to the candidate, including candidate records. The database can include a single database or a collection of databases. In some embodiments, a database management system (e.g., database management system 185 from FIG. 1B) can collect, update, aggregate, and format the collection of data associated with the candidate from among several disparate database servers (e.g., client database 118, service provider database 108, third-party database 122 from FIG. 1A), and the like.

At step 406, the collection of data is evaluated against a plurality of trial criteria. In some embodiments, an eligibility engine can evaluate the collection of data/candidate records against multiple trial criteria to determine if the candidate is eligible or potentially eligible for a trial. The evaluation can be performed by comparing one or more characteristics, questions/answers, test results, etc. for a candidate in the collection of data against one or more criteria for a trial. The evaluation comparison can be performed using any combination of methods. For example, a comparison can be performed using a matching algorithm, artificial intelligence, machine learning, neural network, etc. or a combination thereof. The eligibility can be based on a score, ranking, confidence score, etc. of the determination. The eligibility engine can access an aggregation of candidate records and trial records when making a determination.

At step 408, at least one potential trial for the candidate is determined. The eligibility engine can simultaneously evaluate the collection of data against trial criteria from multiple trials. Based on the evaluation, the eligibility can determine that the candidate is potentially eligible for one, several, or zero trials. The candidate can potentially be eligible for any number of trials provided that the candidate meets certain criteria for each of the trials. Any of the trials for which the candidate is potentially eligible can include local trials (e.g., trials that have a principal investigator that resides in the same state as the candidate) or non-local trials. For example, the candidate can be potentially eligible for a trial that has a headquarters in California, even if the candidate resides across the country in South Carolina. The headquarters of a trial can be defined by a state, city, or region in which the principal investigator resides.

Trial criteria for a trial can include inclusion criteria, exclusion criteria, or a combination of both types of criteria. In some embodiments, the inclusion criteria can take the form of a series of yes or no questions. In some embodiments, for a candidate to pass one of the inclusion criteria, the answer to a yes or no question associated with the inclusion criterion is yes. For example, inclusion criteria for an HIV trial can include the question “has the candidate tested positive for HIV?” Similarly, the exclusion criteria can take the form of yes or no questions and, to pass an exclusion criterion question, the answer will be no. If a candidate fails at least one criterion of a particular trial, the candidate may be deemed currently ineligible for the trial. Although, a candidate may be deemed ineligible for a trial during a present candidate encounter, the eligibility for the candidate can be reevaluated during a future candidate encounter or as a database for trials grows and gets updated.

Substantially contemporaneous to determining the at least one potential trial, information regarding the at least one trial can be presented. At step 410, a trial tab within the GUI is generated. In some embodiments, a new trial GUI is created instead of the trial tab. The trial tab can be trials tab 202 depicted in FIGS. 2A-2C. In some embodiments the trial tab can be initially generated as a blank page.

At step 412, information regarding the at least one potential trial can be automatically populated within the trial tab. The eligibility engine can generate customizable graphical representations showing information about a trial and how a candidate is a match, is not a match, or potentially is a match to be eligible for that trial. The graphical representations can be populated within the trial tab as potential trials are determined for the candidate. For example, the eligibility engine can generate the trial tab containing one or more display sections showing an individual data set, an aggregation of historical data sets, a collection of aggregated historical data sets, etc. The data sets can include any combination of data. For example, the data sets can be related to medical data for the candidate, test data for the candidate, biographical information, etc. which can include test results, biometric data, amount of inclusion criteria met/not met, amount of exclusion criteria met/not met, characteristics of trials, etc. The data sets can also include supplemental data that correspond to the data sets.

The graphical representations can also include prompts to be delivered to solicit additional information about the candidate to make further determinations about candidate eligibility. For example, the eligibility engine can generate a trial tab containing one or more display sections providing prompts to a trial coordinator to ask questions to the candidate, receiving feedback from the candidate, receiving election(s)/rejection(s) from the candidate for participation within the trial, etc. The eligibility engine can review, interpret, analyze, modify and/or determine how to best visualize the data.

In some embodiments, the trial tab can include a first portion, such as first portion 200 depicted in FIG. 2A. Graphical elements included in the upper portion can include a list of trial opportunities. Each trial opportunity in the list can be characterized by columns with column titles including ‘trial title’, ‘therapeutic area’, ‘trial phase’, and/or ‘completeness’.

The physician can select a specific trial opportunity from the list by tapping on any location in a row for the specific trial within the list. Once the specific trial opportunity is selected, a ‘Trial Details’ graphical element can be revealed within the ‘Trials’ tab. The ‘Trial Details’ graphical element can include information to assist the physician to learn about the trial opportunity and help the physician explain the trial opportunity to the candidate.

In some embodiments, the trial tab can include a lower left portion that summarizes inclusion criteria for the selected trial and a lower right portion that summarizes exclusion criteria for the selected trial. The summary of inclusion criteria (and exclusion criteria) can be presented as a list of yes or no questions. Each question in the list of yes or no questions can be characterized as ‘yes’, ‘no’, or ‘unanswered’. The physician can assist with the list of yes or no questions of the summary of inclusion criteria. The physician can provide additional information regarding qualifications of the candidate by submitting answers to unanswered questions, confirm or correct answered questions, or provide any of the yes or no questions to the candidate.

At step 412, for each potential trial, a determination can be made as to whether the candidate would like to be considered for the potential trial. If the candidate is still potentially eligible for a particular trial, even after the physician assists with determining answers to a portion of unanswered questions in the summaries of inclusion/exclusion criteria, the physician can ask if the candidate is interested in the particular trial.

At step 414, after determining that a candidate is interested in the potential trial, information regarding the at least one potential trial can be exported. The physician can use the trial tab to provide a response to a question “Is the candidate interested in this trial?” displayed on the trial tab. Examples of the response can include yes, no, etc. When the physician selects the yes response, the physician can refer the candidate to the trial. The physician can refer the candidate to the trial and maintain a position as specialist or primary physician to the candidate. In some non-limiting embodiments, the candidate can choose to participate in the trial under supervision of a separate provider without changing their preferred physicians or specialists. In some embodiments, and at step 416, after selecting yes to the “Is the candidate interested in this trial?” question, the information regarding the potential trial can automatically be exported to a trial candidate GUI. The information regarding the potential trial can include candidate biographical information such as first and last name, date of birth, medical record number, home phone number, home address, mobile phone number, etc. The information can also include qualifications of the candidate relevant to the potential trial, such as a completion score associated with the inclusion and exclusion criteria.

At step 418, after determining that a candidate is not interested in the potential trial, information regarding the potential trial is removed the trial tab. The potential trial can be removed from the list on the trial tab. The candidate may no longer be considered for the potential trial. The collection of data associated with the candidate may be updated upon determining that the candidate is not interested in the potential trial, and/or may be updated based on a response from the candidate.

As noted herein, the flowchart of FIG. 4 illustrates the architecture, functionality, and operation of possible implementations of systems, methods, and computer program products according to various embodiments. In this regard, each block in the flowchart or block diagrams may represent a module, segment, or portion of code, which includes one or more executable instructions for implementing the specified logical functions. It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combination of blocks in the block diagrams and/or flowchart illustration, can be implemented by special purpose hardware-based systems that perform the specified functions or acts, or combinations of special purpose hardware and computer instructions.

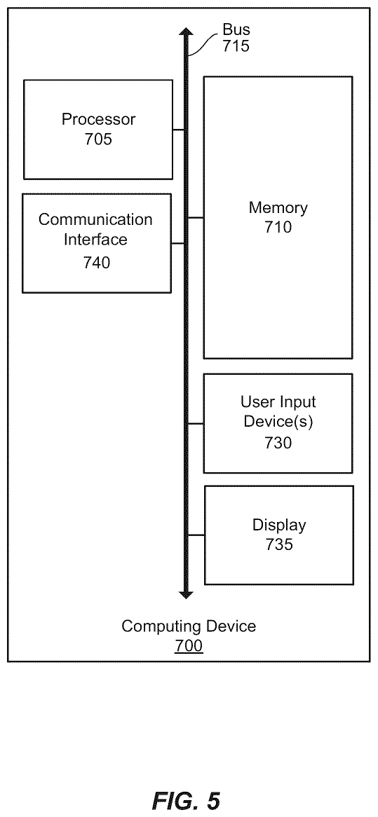

FIG. 5 illustrates an example of a computing device 700 suitable for use with systems and methods for generating a trials tab or a candidate list GUI, according to this disclosure. The computing device 700 includes a processor 705 which is in communication with the memory 710 and other components of the computing device 700 using one or more communications buses 715. The processor 705 is configured to execute processor-executable instructions stored in the memory 710 to perform one or more methods according to different examples, such as part or all of the process 400 described above with respect to FIG. 4. In this example, the memory 710, which may be a non-transitory computer readable medium, stores processor-executable instructions that provide the various components and their related functionality, as discussed above with respect to FIGS. 1A-4. The computing device 700, in this example, also includes one or more user input devices 730, such as a keyboard, mouse, touchscreen, microphone, etc., to accept user input. The computing device 700 also includes a display 735 to provide visual output to a user such as a user interface.

The computing device 700 also includes a communications interface 740. In some examples, the communications interface 740 may enable communications using one or more networks, including a area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

While some examples of methods and systems herein are described in terms of software executing on various machines, the methods and systems may also be implemented as specifically-configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory (e.g., a non-transitory computer-readable memory), such as executing one or more computer programs. Such processors may comprise a microprocessor, a digital signal processor (DSP), an algorithm-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, that may store processor-executable instructions that, when executed by the processor, can cause the processor to perform methods according to this disclosure as carried out, or assisted, by a processor. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

### Additional Considerations

Although specific examples have been described, various modifications, alterations, alternative constructions, and equivalents are possible. Examples are not restricted to operation within certain specific data processing environments but are free to operate within a plurality of data processing environments. Additionally, although certain examples have been described using a particular series of transactions and steps, it should be apparent to those skilled in the art that this is not intended to be limiting. Although some flowcharts describe operations as a sequential process, many of the operations may be performed in parallel or concurrently. In addition, the order of the operations may be rearranged. A process may have additional steps not included in the figure. Various features and aspects of the above-described examples may be used individually or jointly.

Further, while certain examples have been described using a particular combination of hardware and software, it should be recognized that other combinations of hardware and software are also possible. Certain examples may be implemented only in hardware, or only in software, or using combinations thereof. The various processes described herein may be implemented on the same processor or different processors in any combination.

Where devices, systems, components or modules are described as being configured to perform certain operations or functions, such configuration may be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory computer readable-memory/medium, or any combination thereof. Processes may communicate using a variety of techniques including but not limited to conventional techniques for inter-process communications, and different pairs of processes may use different techniques, or the same pair of processes may use different techniques at different times.

Specific details are given in this disclosure to provide a thorough understanding of the examples. However, examples may be practiced without these specific details. For example, well-known circuits, processes, algorithms, structures, and techniques have been shown without unnecessary detail in order to avoid obscuring the examples. This description provides example examples only, and is not intended to limit the scope, applicability, or configuration of other examples. Rather, the preceding description of the examples will provide those skilled in the art with an enabling description for implementing various examples. Various changes may be made in the function and arrangement of elements.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific examples have been described, these are not intended to be limiting. Various modifications and equivalents are within the scope of the following claims.

In the foregoing specification, aspects of the disclosure are described with reference to specific examples thereof, but those skilled in the art will recognize that the disclosure is not limited thereto. Various features and aspects of the above-described disclosure may be used individually or jointly. Further, examples may be utilized in any number of environments and applications beyond those described herein without departing from the broader spirit and scope of the specification. The specification and drawings are, accordingly, to be regarded as illustrative rather than restrictive.

In the foregoing description, for the purposes of illustration, methods were described in a particular order. It should be appreciated that in alternate examples, the methods may be performed in a different order than that described. It should also be appreciated that the methods described above may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, floppy diskettes, ROMs, RAMS, EPROMs, EEPROMs, magnetic or optical cards, flash memory, or other types of machine-readable mediums suitable for storing electronic instructions. Alternatively, the methods may be performed by a combination of hardware and software.

Where components are described as being configured to perform certain operations, such configuration may be accomplished, for example, by designing electronic circuits or other hardware to perform the operation, by programming programmable electronic circuits (e.g., microprocessors, or other suitable electronic circuits) to perform the operation, or any combination thereof.

While illustrative examples of the application have been described in detail herein, it is to be understood that the inventive concepts may be otherwise variously embodied and employed, and that the appended claims are intended to be construed to include such variations, except as limited by the prior art.

What is claimed is:

1. A computer-implemented method comprising:
receiving a request related to a candidate;
accessing a database that includes a collection of data related to the candidate;
receiving an update to the collection of data to generate an updated collection of data;
automatically evaluating the updated collection of data against a plurality of trial criteria;
determining at least one potential trial for the candidate based on the evaluation;
converting the updated collection of data into one or more criteria sections;
automatically generating, substantially contemporaneous to determining the at least one potential trial, a graphical user interface (GUI) including the one or more criteria sections; and
automatically displaying, within the GUI, a message to one or more users related to eligibility of the candidate for the at least one potential trial.

2. The method of claim 1, wherein automatically generating the GUI further comprises:
generating, by a processor, a trials dashboard tab within the GUI; and
populating, automatically by the processor substantially contemporaneous to determining the at least one potential trial, the one or more criteria sections regarding the at least one potential trial on the trials dashboard tab.

3. The method of claim 2, wherein evaluating the updated collection of data against the plurality of trial criteria comprises producing, for each of the at least one potential trial, a completion score for the candidate, the method further comprising:
receiving, via the trials dashboard tab and by the processor, additional information regarding qualifications of the candidate relevant to criteria for the at least one potential trial; and
updating, by the processor, the completion score associated with the at least one potential trial.

4. The method of claim 1, further comprising receiving, for each of the at least one potential trial, a response regarding a candidate interest in a potential trial.

5. The method of claim 4, for each response that indicates that the candidate is not interested in the potential trial, the method further comprises removing, by a processor, the potential trial associated with the response; and
updating, by the processor, the updated collection of data with the response regarding the candidate interest in the potential trial.

6. The method of claim 4, for each response that indicates that the candidate is interested in the potential trial, the method further comprising exporting, by a processor, the updated collection of data regarding the potential trial associated with the response into a trial candidate module.

7. The method of claim 6, further comprising importing, automatically by the processor, additional information regarding the potential trial into the trial candidate module, the additional information related to an available candidate that has expressed interest in at least one other trial similar to the potential trial.

8. A system comprising:
one or more processors; and
one or more memories that include instructions executable by the one or more processors to perform operations, the operations comprising:
receiving a request related to a candidate;
accessing a database that includes a collection of data related to the candidate;
receiving an update to the collection of data to generate an updated collection of data;
automatically evaluating, the updated collection of data against a plurality of trial criteria;
determining at least one potential trial for the candidate based on the evaluation;
converting the updated collection of data into one or more criteria sections;
automatically generating, substantially contemporaneous to determining the at least one potential trial, a graphical user interface (GUI) including the one or more criteria sections; and
automatically displaying, within the GUI, a message to one or more users related to eligibility of the candidate for the at least one potential trial.

9. The system of claim 8, wherein the operation of automatically generating the GUI further comprises:
generating a trials dashboard tab within the GUI; and
populating, automatically substantially contemporaneous to determining the at least one potential trial, the one or more criteria sections regarding the at least one potential trial on the trials dashboard tab.

10. The system of claim 9, wherein evaluating the updated collection of data against the plurality of trial criteria comprises producing, for each of the at least one potential trial, a completion score for the candidate, the operations further comprising:
receiving, via the trials dashboard tab, additional information regarding qualifications of the candidate relevant to criteria for the at least one potential trial; and
updating the completion score associated with the at least one potential trial.

11. The system of claim 8, the operations further comprising:
receiving, for each of the at least one potential trial, a response regarding candidate interest in a potential trial.

12. The system of claim 11, for each response that indicates that the candidate is not interested in the potential trial, the operations further comprises:
removing the potential trial associated with the response; and
updating the updated collection of data with the response regarding the candidate interest in the potential trial.

13. The system of claim 11, for each response that indicates that the candidate is interested in the potential trial, the operations further comprise:
exporting the updated collection of data regarding the potential trial associated with the response into a trial candidate module.

14. The system of claim 13, the operations further comprise:
importing, automatically, additional information regarding a potential trial into the trial candidate module, the additional information related to an available candidate that has expressed interest in at least one other trial similar to the potential trial.

15. A non-transitory computer-readable memory storing a plurality of instructions executable by one or more processors for causing the one or more processors to perform operations comprising:
receiving a request related to a candidate;
accessing a database that includes a collection of data related to the candidate;
receiving an update to the collection of data to generate an updated collection of data;
automatically evaluating the updated collection of data against a plurality of trial criteria;
determining at least one potential trial for the candidate based on the evaluation;
converting the updated collection of data into one or more criteria sections;
automatically generating, substantially contemporaneous to determining the at least one potential trial, a graphical user interface (GUI) including the one or more criteria sections; and
automatically displaying, within the GUI, a message to one or more users related to eligibility of the candidate for the at least one potential trial.

16. The non-transitory computer-readable memory of claim 15, wherein the operation of automatically generating the GUI further comprises:
generating a trials dashboard tab within the GUI; and
populating, automatically substantially contemporaneous to determining the at least one potential trial, the one or more criteria sections regarding the at least one potential trial on the trials dashboard tab.

17. The non-transitory computer-readable memory of claim 16, wherein evaluating the updated collection of data against the plurality of trial criteria comprises producing, for each of the at least one potential trial, a completion score for the candidate, the operations further comprising:
receiving, via the trials dashboard tab, additional information regarding qualifications of the candidate relevant to criteria for the at least one potential trial; and
updating the completion score associated with the at least one potential trial.

18. The non-transitory computer-readable memory of claim 15, the operations further comprising receiving, for each of the at least one potential trial, a response regarding a candidate interest in a potential trial.

19. The non-transitory computer-readable memory of claim 18, for each response that indicates that the candidate is not interested in the potential trial, the operations further comprises:
removing the potential trial associated with the response; and
updating the updated collection of data with the response regarding the candidate interest in the potential trial.

20. The non-transitory computer-readable memory of claim 18, for each response that indicates that the candidate is interested in the potential trial, the operations further comprise: exporting the updated collection of data regarding the potential trial associated with the response into a trial candidate module.
